(19) United States
(12) Patent Application Publication
(10) Pub. No.: US 2026/0202689 A1
(43) Pub. Date: Jul. 16, 2026

(54) METHOD AND OPTICAL SYSTEM FOR DETECTING A PUPIL OF AN EYE LOCATED WITHIN A DETECTION REGION USING A MACHINE LEARNING ALGORITHM AND DATA GLASSES COMPRISING THE OPTICAL SYSTEM

(71) Applicant: Robert Bosch GmbH, Stuttgart (DE)

(72) Inventors: Benjamin Cramer, Leonberg (DE); Johannes Meyer, Haseluenne (DE)

(21) Appl. No.: 19/436,474

(22) Filed: Dec. 30, 2025

(30) Foreign Application Priority Data
Jan. 10, 2025 (DE) 10 2025 100 735.7

(51) Int. Cl. G02C 11/00; A61B 3/10; G02B 27/00

(52) U.S. Cl. CPC G02C 11/10; A61B 3/10; G02B 27/0093

(57) ABSTRACT

Methods for detecting a pupil of an eye located within a detection region. The methods include: deflecting a laser beam into a beam direction which is variable in at least one dimension; scanning the detection region by varying the beam direction; detecting an amplitude value of scattered light from the laser beam emitted into the detection region onto the eye using a detector; providing the detected amplitude value and the associated beam direction, or a group of amplitude values with associated beam directions, to a machine learning algorithm, wherein the detected amplitude value and the associated beam direction, or the group of amplitude values with associated beam directions, is provided to the algorithm iteratively; wherein the pupil is detected using the algorithm depending on the iteratively provided amplitude value and the associated beam direction or the group of amplitude values with associated beam directions.

### CROSS REFERENCE

The present application claims the benefit under 35 U.S.C. § 119 of Germany Patent Application No. DE 10 2025 100 735.7 filed on Jan. 10, 2025, which is expressly incorporated herein by reference in its entirety.

### FIELD

The present invention relates to a method and optical system for detecting a pupil of an eye located within a detection region using a machine learning algorithm and data glasses comprising the optical system.

### BACKGROUND INFORMATION

In systems for determining a gaze direction of an eye on the basis of a position of a pupil of the eye, for example, a detection region in which the eye is located is scanned using a laser beam, and scattered light from the laser beam is detected using a detector. A reflectivity map in the form of an image of the detection region is generated based on the detected light and all deflection angles of the scanned detection region. If an eye is located within the detection region, the laser beam hitting the eye will be scattered differently depending on a surface of the eye. This difference is visible in the reflectivity map, from which, in particular, the position of the pupil can be ascertained. For example, the pupil appears bright or dark depending on the arrangement of the detector and a source of the laser beam. If the detector is located outside an optical axis of the laser beam, the pupil acts like an aperture and appears dark in the reflectivity map. If the detector is located on the optical axis of the laser beam, for example in a laser feedback interferometry (LFI) sensor, the laser beam is reflected back due to a high reflectivity of a retina of the eye, resulting in an amplitude modulation which the detector detects. In this arrangement, the pupil appears bright in the reflectivity map. Determining the position of the pupil requires an evaluation of the detected reflectivity map, which constitutes a complete scan of the detection region. Depending on a resolution of the scan, memory requirements increase significantly, as the complete reflectivity map is provided for evaluation. Furthermore, determination of the pupil's position using conventional evaluation methods can only begin once the complete reflectivity map is available.

Therefore, an optical system that enables a gaze direction to be detected, for example based on a pupil position or pupil orientation, and that has reduced memory requirements and low latency is desirable.

### SUMMARY

This may be achieved by a method, an optical system and data glasses according to certain features of the present invention.

According to an example embodiment of the present invention, a method for detecting a pupil of an eye located within a detection region comprises: deflecting a laser beam into a beam direction which is variable in at least one dimension, in particular in two dimensions; scanning the detection region by varying the beam direction; detecting an amplitude value of scattered light from the laser beam emitted into the detection region onto the eye using a detector, wherein the pupil is detectable depending on the amplitude value and the associated beam direction; providing the detected amplitude value and the associated beam direction, or a group of amplitude values with associated beam directions, to a machine learning algorithm, wherein the detected amplitude value and the associated beam direction, or the group of amplitude values with associated beam directions, is provided to the algorithm iteratively, for example, immediately after detection; wherein the pupil is detected using the algorithm depending on the iteratively provided amplitude value and the associated beam direction or the group of amplitude values with associated beam directions.

The detection region can be referred to as a scan eyebox, for example, and spans a region that can be scanned using the laser beam. A size of the detection region is defined by limits or a spread of the beam direction, for example, the maximum possible deflection angles. The size or shape of the detection region can be influenced by optical elements that deflect the laser beam, such as lenses or holographic optical elements (HOE). In the present context, the amplitude value characterizes in particular a brightness of the scattered light. It can be detected directly by the detector or in connection with interference in an illumination device that generates the laser beam. In the present context, the group of amplitude values with associated beam directions describes a set of, in particular successive, amplitude values that was detected for a portion of the detection region. The group of amplitude values does not, in particular, mean a complete scan of the detection region. The detection region can be completely covered, for example, by a large number of groups of amplitude values with associated beam directions. In the present context, the term iterative means, for example, step by step. This is to be understood in particular as meaning that a detected amplitude value or a group of amplitude values with corresponding beam directions is provided to the algorithm as input immediately after detection, in particular in temporal succession. The provided group of amplitude values or the individual amplitude value with the corresponding associated beam directions can be referred to collectively as input below. The term “directly” comprises, in particular, processing, for example digitization and/or filtering, of the detected amplitude value or group of amplitude values; however, it does not comprise, for example, creating a complete image or scan of the detection region, for example in the form of a reflectivity map, which is provided to the algorithm. The amplitude value or group of amplitude values with the associated beam directions is therefore provided to the algorithm online or in-frame, i.e., within or during a complete scanning cycle of the detection region. The algorithm then evaluates such input online, in particular in the form of pupil detection. For example, the pupil is detected for each individual input without the need to detect or provide a complete image of the detection region and the eye located therein. Therefore, in particular, such an image does not need to be saved or generated. This reduces the memory space required. Moreover, a latency time between detecting the amplitude value and detecting the pupil is reduced. In the present context, detection comprises in particular any acquisition of information about the pupil of the eye. Such information includes, for example, a spatial position of the pupil in the detection region, a shape or contour of the pupil, an orientation, parameters of an ellipse of the pupil, and/or a segmentation of the detection region that represents the pupil, similar to an image or a reflectivity map of the detection region.

According to an example embodiment of the present invention, it may be provided for the algorithm to be configured as a neural network, for example using long short-term memory cells or gated recurrent units, wherein the amplitude values and the associated beam direction or the group of amplitude values with associated beam directions of past detection steps are taken into account as hidden states when detecting the pupil, depending on the currently provided amplitude value and the associated beam direction or the currently provided group of amplitude values with associated beam directions. In the present context, a step refers in particular to a detection step, i.e., a step in which the algorithm is provided with an input and, depending on the input, detects the pupil or generates an output. Neural networks can efficiently take into account amplitude values from past steps without requiring additional memory for them. Also, the algorithm can be efficiently adapted to predefined scanning patterns. The algorithm can be trained for different scanning patterns, for example using corresponding data. This increases the quality and reliability of the method.

According to an example embodiment of the present invention, it may be provided for detection to comprise estimating a segmentation of the pupil in the detection region and/or estimating a position of the pupil, for example a geometric centroid or center of the pupil, and/or estimating a contour, position and/or orientation of the pupil, for example in the form of ellipse parameters. Estimating the ellipse parameters reduces the memory space required, since they can be provided, for example, by means of a vector that characterizes, for example, a position of an ellipse center in the detection region and a position or orientation of the major and minor axis. A result of detecting or estimating pupil information, as mentioned above, can be referred to as the output of the algorithm. In particular, the algorithm estimates an output for each input provided.

According to an example embodiment of the present invention, it may be provided to use a regression layer in the algorithm to estimate the contour, position and/or orientation of the pupil, for example in the form of the ellipse parameters. The regression layer enables efficient and reliable ascertaining or estimation of the ellipse parameters.

According to an example embodiment of the present invention, it may be provided for the group of amplitude values with associated beam directions to comprise a predefined sequence of beam directions, for example, beam directions of individual scanned rows and/or columns of the detection region. Providing the algorithm with input in a repeating pattern or rhythm improves the algorithm's reliability.

According to an example embodiment of the present invention, it may be provided for the beam direction to be defined by a first deflection angle and/or a second deflection angle, for example tilt angles or scan axes of at least one micro-electro-mechanical system (MEMS) mirror. The deflection angles allow the beam direction to be clearly defined. Moreover, the MEMS mirrors allow for flexible and efficient variation of the beam direction by varying the deflection angles.

According to an example embodiment of the present invention, it may be provided for the algorithm to comprise a temporal convolutional neural network and/or a transformer model architecture and/or a hidden Markov model.

According to an example embodiment of the present invention, an optical system for detecting a pupil of an eye located within a detection region comprises: an illumination device configured to generate a laser beam, in particular an infrared laser beam; a deflection device configured to deflect the laser beam into a beam direction which is variable in at least one dimension, in particular in two dimensions, and to scan the detection region by varying the beam direction; a detector configured to detect an amplitude value of scattered light from the laser beam emitted into the detection region onto the eye, wherein the pupil is detectable depending on the amplitude value and the associated beam direction; wherein the optical system is configured to iteratively, for example immediately after detection, provide the detected amplitude value and the associated beam direction, or a group of amplitude values with associated beam directions, to a machine learning algorithm, wherein the optical system comprises a computing device configured to detect the pupil using the algorithm depending on the iteratively provided amplitude value and an associated beam direction or the iteratively provided group of amplitude values with associated beam directions.

It may be provided for the optical system to be configured to carry out a method according to the above-described implementation of the present invention.

The data glasses comprise the optical system according to the above-described implementation of the present invention.

Further embodiments of the present invention can be found in the figures and the following description.

### BRIEF DESCRIPTION OF THE DRAWINGS

FIG. 1 shows a flowchart of a method for detecting a pupil, according to an example embodiment of the present invention.

FIG. 2 shows a schematic representation of an optical system for detecting the pupil, according to an example embodiment of the present invention.

FIG. 3 shows a schematic representation of amplitude values.

FIG. 4 shows a schematic representation of a course of the method according to an example embodiment of the present invention.

FIG. 5 shows a schematic representation of data glasses comprising the optical system, according to an example embodiment of the present invention.

### DETAILED DESCRIPTION OF EXAMPLE EMBODIMENTS

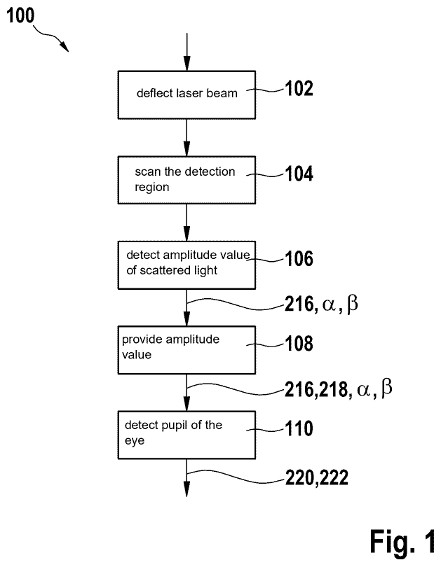

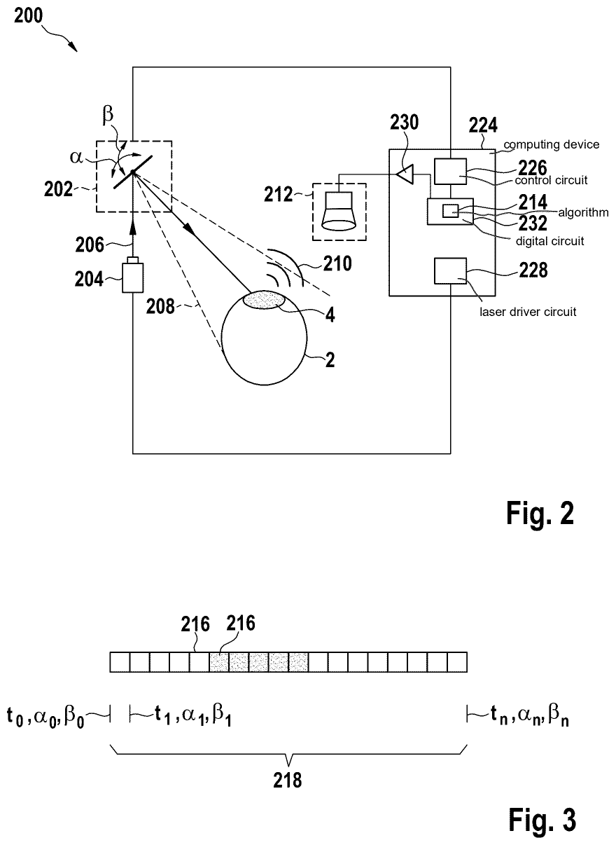

FIG. 1 shows a flowchart of a method 100 for detecting 110 a pupil 4 of an eye 2 located within a detection region 208, and FIG. 2 shows a schematic representation of an optical system 200 for detecting 110 the pupil 4 of the eye 2 located within the detection region 208. The method 100 comprises deflecting 102 a laser beam 206 into a beam direction α, β which is variable in at least one dimension, in particular in two dimensions, and scanning 104 the detection region 208 by varying the beam direction α, β. The beam direction α, β can be varied, for example, in a predefined pattern, such as a Lissajous pattern. The laser beam is, for example, an infrared laser beam, in particular having a wavelength of 850 nm or 940 nm or an alternative wavelength in the infrared range. This is referred to as scanning 104. The term “complete scan” describes a complete scanning of the detection region 208. The detection region 208 can, for example, be scanned discretely. The beam direction α, β can be varied in discrete steps. A region of the detection region 208 that is scanned by a beam direction α, β, or that can be assigned to a single beam direction α, β, can be referred to as a pixel of the detection region 208.

The method 100 comprises detecting 106 an amplitude value 216 of scattered light 210 from the laser beam 206 emitted into the detection region 208 onto the eye 2 using a detector 212, wherein the pupil 4 is detectable depending on the amplitude value 216 and the associated beam direction α, β. A scattering behavior of the incident laser beam 206 varies depending on the point where the laser beam 206 hits the eye 2. The amplitude value 216 characterizes, for example, a brightness or intensity of the scattered light 210. Thus, a corresponding amplitude value 216 is detected for example for each pixel of the detection region 208 or for each beam direction α, β.

The method 100 comprises providing 108 the detected amplitude value 216 (FIG. 3) and the associated beam direction α, β, or a group 218 (FIG. 3) of amplitude values 216 with associated beam directions α, β, to a machine learning algorithm 214, wherein the detected amplitude value 216 and the associated beam direction α, β, or the group 218 of amplitude values 216 with associated beam directions α, β, is provided to the algorithm 214 iteratively, for example immediately after detection 106. Such provision 108 takes place in particular in temporal succession. The beam direction can also be implicitly contained in a provided sequence. The group 218 of amplitude values 216 is formed, for example, from individual amplitude values 216 and their associated beam direction, wherein they are temporarily stored to form the group 218. Providing 108 an input in the form of amplitude values 216 or the group 218 of amplitude values with the associated beam directions α, β to the machine learning algorithm 214 can also be referred to as injection.

In the method 100, the pupil 4 is detected 110 using the algorithm 214 depending on the iteratively provided amplitude value 216 and the associated beam direction α, β or the group 218 of amplitude values 216 with associated beam directions α, β. Such detection 110 thus takes place online or in-frame, i.e., during an ongoing scanning of the detection region 208. In particular, as a result of such detection 110, an output is generated by the algorithm 214 for each input.

The optical system 200 for detecting 110 a pupil 4 of the eye 2 located within the detection region 208 comprises an illumination device 204 configured to generate the laser beam 206, in particular the infrared laser beam. The optical system 200 comprises a deflection device 202 configured to deflect the laser beam 206 into the beam direction α, β which is variable in at least one dimension, in particular in two dimensions, and to scan the detection region 208 by varying the beam direction α, β. The deflection device 202 can be configured for example as a MEMS mirror or comprise a large number of MEMS mirrors that are tiltable in one dimension.

The optical system 200 comprises the detector 212, which is configured to detect 106 the amplitude value 216 of scattered light 210 from the laser beam 206 emitted into the detection region 208 onto the eye 2, wherein the pupil 2 is detectable depending on the amplitude value 216 and the associated beam direction α, β. The detector 212 can be provided for example in the illumination device 204, wherein the illumination device 204 is configured, for example, as a laser feedback interferometry sensor.

The optical system 200 is configured to iteratively, for example immediately after detection 106, provide 108 the detected amplitude value 216 and the associated beam direction α, β, or the group 218 of amplitude values 216 with their respective associated beam directions α, β, to the machine learning algorithm 214. The optical system 200 comprises a computing device 224, which is configured to detect 110 the pupil 4 using the algorithm 214 depending on the iteratively provided amplitude value 216 and the associated beam direction α, β or the iteratively provided group 218 of amplitude values 216 with associated beam directions α, β.

It may be provided for the computing device 224 to comprise a control circuit 226 configured to control the deflection device 202, and to comprise a laser driver circuit 228 configured to control the illumination device 204. The computing device 224 can moreover comprise a processing circuit 230, which is configured, for example, to digitize, filter and process the detected amplitude value 216 and/or to form the group 218 of amplitude values 216. It may be provided for the algorithm 214 to be implemented on a digital circuit 232 of the computing device 224. It is possible for the computing device 224 to be configured as a system-on-chip. The optical system 200 is generally configured to carry out the method 100.

It may be provided for the algorithm 214 to be configured as a neural network, for example using long short-term memory (LSTM) cells or gated recurrent units (GRU), wherein the amplitude values 216 and the associated beam direction α, β or the group 218 of amplitude values 216 with associated beam directions α, β of past detection 110 steps are taken into account as hidden states when detecting 110 the pupil 4, depending on the currently provided amplitude value 216 and the associated beam direction α, β or the currently provided group 218 of amplitude values 216 with associated beam directions α, β.

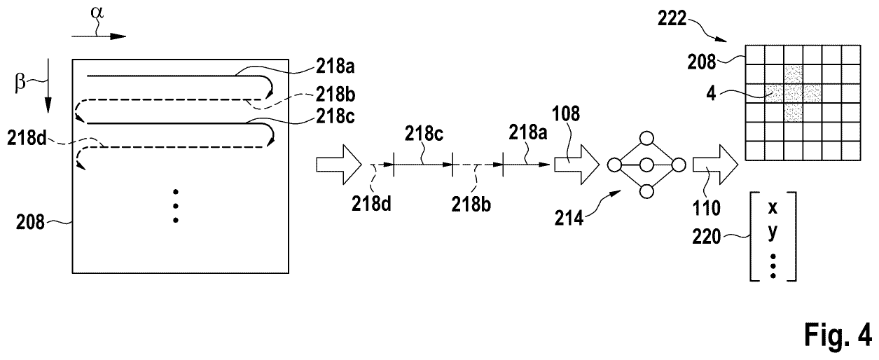

It may be provided for detection 110 to comprise estimating a segmentation 222 (FIG. 4) of the pupil 4 in the detection region 208 and/or estimating a position of the pupil 4, for example a geometric centroid or center of the pupil 4, and/or estimating a contour, position and/or orientation of the pupil 4, for example in the form of ellipse parameters 220 (FIG. 4). The ellipse parameters 220 can, for example, be estimated in the form of a vector that describes the position and/or orientation of the ellipse. The vector can, for example, comprise coordinates of the position of a center of the ellipse, parameters of a major and minor axis of the ellipse, and parameters of an orientation of the ellipse. The vector can moreover comprise, for example, an estimated error that characterizes a reliability of detection 110, or of estimated information of the pupil 4.

It may be provided to use a regression layer in the algorithm 214 to estimate the contour, position and/or orientation of the pupil 4, for example in the form of the ellipse parameters 220. The above-mentioned estimation can, for example, also be based on a previously estimated segmentation 222 of the pupil 4 in the detection region. The algorithm 214 can moreover be configured to directly estimate the ellipse parameters 220 as output.

It may be provided for the beam direction α, β to be defined by a first deflection angle α and/or a second deflection angle β, for example tilt angles or scan axes of at least one micro-electro-mechanical system mirror.

FIG. 3 shows a schematic representation of amplitude values 216 or a group 218 of amplitude values. FIG. 3 shows a discretized, for example digitized, time series of amplitude values 216. Each time step t₀, t₁ to tₙ can be assigned a corresponding beam direction α₀, α₁ to αₙ and β₀, β₁ to βₙ and a corresponding amplitude value 216. It is possible for the relevant beam direction α, β to be implicitly provided to the algorithm 214 by providing the corresponding time step t₀, t₁ to tₙ. The algorithm 214 is therefore designed to detect the corresponding beam direction α, β based on the provided or present time step t₀, t₁ to tₙ. This can be achieved, for example, by having the algorithm 214 learn the pattern in which the beam direction α, β is varied. Multiple amplitude values 216 form a group 218 of amplitude values 216. In the example shown, pixels of the detection region 208 are shown in dark, for the beam direction α, β of which the laser beam 206 is emitted onto the pupil 4 of the eye 2.

FIG. 4 shows a schematic course of the method 100. It may be provided for the group 218 of amplitude values 216 with associated beam directions α, β to comprise a predefined sequence of beam directions α, β, for example, beam directions α, β of individual scanned rows and/or columns of the detection region 208. In the example shown, the detection region 208 is scanned row by row using the deflection angles α and β, which define the beam direction α, β. Alternative scanning patterns are also possible, in which, for example, the MEMS mirrors are operated resonantly. In the example shown, each row represents a group 218a to 218d, which is composed of individual amplitude values 216. The groups 218a to 218d of amplitude values 216 are provided to the algorithm 214 iteratively or sequentially. The algorithm 214 estimates or detects 110, for example, the segmentation 222 and/or the ellipse parameters 222 of the pupil 4 sequentially for each individual provided group 218a to 218d and provides such output accordingly. This means that an output is estimated for example for group 218a, and based on this estimate, for example hidden states are generated in the algorithm 214, which are used when estimating the output based on the group 218b that follows group 218a. This procedure is continued for all subsequent groups 218 of amplitude values 216. The example shown illustrates a course of the method 100, in which groups 218 of amplitude values 216 are provided to the algorithm 214. However, this example is applicable accordingly, with appropriate adjustment, in case that individual amplitude values 216 are directly provided to the algorithm 214. For example, for each amplitude value 216 provided, the segmentation 222 and/or the ellipse parameters 220 are estimated using the algorithm, and the hidden states are adapted for subsequent amplitude values 216.

It may be provided for the algorithm 214 to comprise a temporal convolutional neural network and/or a transformer model architecture and/or a hidden Markov model.

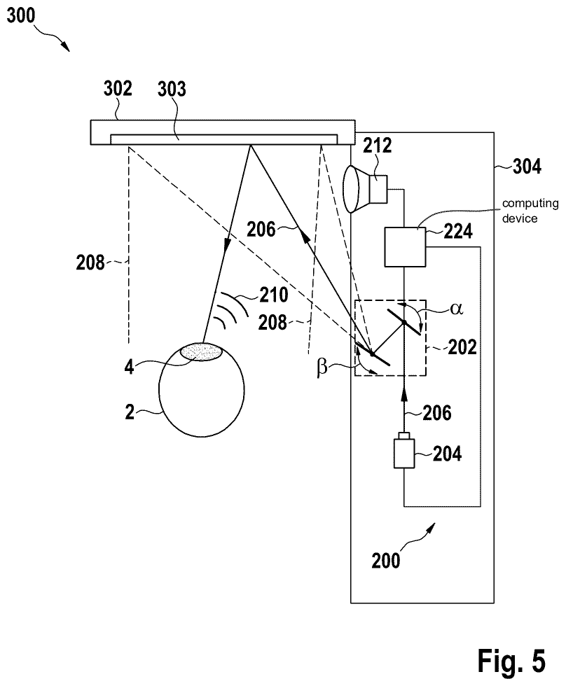

FIG. 5 shows a schematic representation of data glasses 300 comprising the optical system 200 according to the above-described implementation. In the example shown, the data glasses 300 comprise a lens 302 and a temple 304. The lens 302 comprises, for example, a holographic optical element 303, which is configured to redirect the laser beam 206, deflected in the beam direction α, β, onto the eye 2. It is possible for the optical system 200 to be arranged at least partially in the temple 304. Detection 106 of the amplitude values 216 can, for example, be referred to as a flying spot laser scan. The pupil 4 detected using the method 100 or the optical system 200 can be used as the basis of a gaze direction detection system, in particular in the data glasses 300.

What is claimed is:

1. A method for detecting a pupil of an eye located within a detection region, the method comprising the following steps:
deflecting a laser beam into a beam direction which is variable in at least one dimension;
scanning the detection region by varying the beam direction;
detecting an amplitude value of scattered light from the laser beam emitted into the detection region onto the eye using a detector, wherein the pupil is detectable depending on the amplitude value and an associated beam direction;
providing to a machine learning algorithm: (i) the detected amplitude value and the associated beam direction, or (ii) a group of amplitude values with associated beam directions, wherein the detected amplitude value and the associated beam direction, or the group of amplitude values with the associated beam directions, is provided to the algorithm iteratively, immediately after detection;
wherein the pupil is detected using the algorithm depending on the iteratively provided amplitude value and the associated beam direction, or the group of amplitude values with the associated beam directions.

2. The method according to claim 1, wherein the algorithm is configured as a neural network, using long short-term memory cells or gated recurrent units, wherein the amplitude value and the associated beam direction, or the group of amplitude values with the associated beam directions, of past detection steps are taken into account as hidden states when detecting the pupil, depending on a currently provided amplitude value and an associated beam direction or currently provided group of amplitude values with associated beam directions.

3. The method according to claim 1, wherein the detection includes: (i) estimating a segmentation of the pupil in the detection region, and/or (ii) estimating a position of the pupil, and/or (iii) estimating a contour, position and/or orientation of the pupil.

4. The method according to claim 3, wherein a regression layer in the algorithm is used to estimate the contour, position and/or orientation of the pupil, in the form of ellipse parameters.

5. The method according to claim 1, wherein the group of amplitude values with the associated beam directions includes a predefined sequence of beam directions of individual scanned rows and/or columns of the detection region.

6. The method according to claim 1, wherein the beam direction is defined by a first deflection angle and/or a second deflection angle.

7. The method according to claim 1, wherein the beam direction is defined by a tilt angle or scan axis of at least one micro-electro-mechanical system mirror.

8. The method according to claim 1, wherein the algorithm includes a temporal convolutional neural network and/or a transformer model architecture and/or a hidden Markov model.

9. An optical system for detecting a pupil of an eye located within a detection region, the optical system comprising:
an illumination device configured to generate a laser beam;
a deflection device configured to deflect the laser beam into a beam direction which is variable in at least one dimension, and to scan the detection region by varying the beam direction;
a detector configured to detect an amplitude value of scattered light from the laser beam emitted into the detection region onto the eye, wherein the pupil is detectable depending on the amplitude value and an associated beam direction;
wherein the optical system is configured to iteratively, immediately after detection, provide to a machine learning algorithm: (i) the detected amplitude value and the associated beam direction, or (ii) a group of amplitude values with associated beam directions, wherein the optical system includes a computing device configured to detect the pupil using the algorithm depending on the iteratively provided amplitude value and the associated beam direction, or the iteratively provided group of amplitude values with the associated beam directions.

10. The system according to claim 9, wherein the laser beam is an infrared laser beam.

11. The optical system according to claim 9, wherein the optical system is configured to carry out a method including the following steps:
deflecting the laser beam into the beam direction which is variable in the at least one dimension;
scanning the detection region by varying the beam direction;
detecting an amplitude value of scattered light from the laser beam emitted into the detection region onto the eye using the detector;
providing to the machine learning algorithm: (i) the detected amplitude value and the associated beam direction, or (ii) the group of amplitude values with associated beam directions, wherein the detected amplitude value and the associated beam direction, or the group of amplitude values with the associated beam directions, is provided to the algorithm iteratively, immediately after detection;
wherein the pupil is detected using the algorithm depending on the iteratively provided amplitude value and the associated beam direction, or the group of amplitude values with the associated beam directions.

12. Data glasses, comprising:
an optical system for detecting a pupil of an eye located within a detection region, the optical system including:
an illumination device configured to generate a laser beam,
a deflection device configured to deflect the laser beam into a beam direction which is variable in at least one dimension, and to scan the detection region by varying the beam direction,
a detector configured to detect an amplitude value of scattered light from the laser beam emitted into the detection region onto the eye, wherein the pupil is detectable depending on the amplitude value and an associated beam direction,
wherein the optical system is configured to iteratively, immediately after detection, provide to a machine learning algorithm: (i) the detected amplitude value and the associated beam direction, or (ii) a group of amplitude values with associated beam directions, wherein the optical system includes a computing device configured to detect the pupil using the algorithm depending on the iteratively provided amplitude value and the associated beam direction, or the iteratively provided group of amplitude values with the associated beam directions.
